(19) United States
(12) Patent Application Publication
(10) Pub. No.: US 2006/0200248 A1
(43) Pub. Date: Sep. 7, 2006

(54) Prosthesis for the glenoid cavity of the scapula

(76) Inventors: Laurent Beguin, Irigny (FR); Denis Bouttens, Saint Josse (FR); Charles Cohn, Les Adrets de I'Esterel (FR); Geert Declercq, Schilde (BE); Yann Brunnarius, Saint Peray (FR); Jean Grimberg, Le Chesnay (FR); Dominique Huguet, Orvault (FR); Eric Lesprit, Bordeaux (FR); Pierre Massart, Tresserve (FR); Bruno Rio, Nancy (FR); Jacques Teissier, Castelnau Le Lez (FR); Bruno Toussaint, Annecy Le Vieux (FR); Bruno Zipoli, Hossegor (FR)

Correspondence Address: BURR & BROWN, PO BOX 7068, SYRACUSE, NY, 13261-7068, US

(21) Appl. No.: 11/071,539

(22) Filed: Mar. 3, 2005

(51) Int. Cl. A61F 2/40

(52) U.S. Cl. 623/19.11

(57) ABSTRACT

This prosthesis for the glenoid cavity of the scapula comprises a seat, intended to be fixed to the glenoid cavity of the patient to be treated by means of anchoring screws, said seat comprising a plate able to receive a male insert, and said seat being able to receive a female insert, said inserts being intended to cooperate with the humeral head of the shoulder joint in question or with a humeral implant fitted in the area of said joint. The outer face of the peripheral rim, with which the seat is provided, is configured as a truncated morse cone able to cooperate with a male insert provided with a bearing surface of complementary shape. The plate of the seat is provided with means intended to cooperate with a female insert in order to ensure that the latter is fixed by clipping into it.

The invention concerns a prosthesis for the glenoid cavity of the scapula and relates generally to shoulder prostheses.

Depending on the nature of the pathology affecting a patient's shoulder (pain, arthrosis, polyarthritis, tendon or muscle problems, trauma, etc.), it is sometimes necessary to fit a prosthesis of the glenoid cavity, especially when said glenoid cavity has been destroyed partially or completely, but also when a rupture of the rotator cuff is observed.

Traditionally, this prosthesis is in the form of a seat which is fixed in said glenoid cavity of the scapula, or at least what remains of this. Such a glenoid cavity is described, for example, in document FR-A-2 683 142.

The glenoid cavity prostheses known at present are intended to receive an insert which is intended to cooperate with the humeral head or with a humeral implant, and they are thus in the form of a concavity of substantially complementary shape generally not congruent with said humeral head or with said humeral implant. In other words, the insert is generally a female component.

However, depending on the pathology observed, it may sometimes be necessary to use not a female component, but instead a male component, which is then in the form of a spherical or substantially spherical head fixed on the seat of the glenoid cavity prosthesis. In this case, it is therefore generally necessary to employ another glenoid cavity prosthesis in order to permit use of such a male insert.

In other words, the prosthesis in question does not provide the modular character which is desired by practitioners, but which is also desirable from the economic point of view.

Document FR-A-2 652 498 has described a total shoulder prosthesis having a certain modular character. In this document, both the humeral implant and the glenoid cavity prosthesis comprise complementary elements which provide for this modular character. As regards the glenoid cavity prosthesis, these elements are fixed to the periphery of the seat, in particular by cooperation of an internal thread on the insert and an external thread on the seat. However, in use, this mode of fixation is found to be unsatisfactory on account of the risks of micro-movements which may occur and which may thus cause risks of loosening of the glenoid cavity prosthesis. In parallel, in the movements of the articulation in question, wear is observed in the glenoid cavity bone in the area of which said prosthesis is implanted, inherent to the implant, resulting in a rupturing of the fixation means of the seat constituting the implant.

The object of the present invention is to make available a prosthesis for the glenoid cavity of the scapula which also has this modular character and overcomes the disadvantages mentioned above.

This prosthesis for the glenoid cavity of the scapula comprises a seat, intended to be fixed to the glenoid cavity of the patient to be treated by means of anchoring screws, said seat comprising a plate able to receive a female insert, and said seat being able to receive a male insert, said inserts being intended to cooperate with the humeral head of the shoulder joint in question or with a humeral implant fitted in the area of said joint.

This prosthesis is characterized:
in that the outer face of the peripheral rim, with which the seat is provided, is configured as a truncated morse cone able to cooperate with a male insert provided with a bearing surface of complementary shape,
and in that the plate of the seat is provided with means intended to cooperate with a female insert in order to ensure that the latter is fixed by clipping into it.

According to the invention, the seat is provided with two through-holes, each of them inclined by an angle value of between 20° and 35° relative to the general plane of the plate, and being intended to permit the passage of anchoring screws into the glenoid cavity.

According to the invention, the face of the seat, intended to come into contact with the bone of the glenoid cavity, is bulged and is provided with a stem intended to be implanted in said glenoid cavity.

In addition, this same face advantageously has two flat areas situated at end positions relative to the bulged part, and thus situated on either side of the bulged zone and intended to bear inside the glenoid cavity, after suitable reaming. This being so, these flat areas provide a stabilizing function for the prosthesis, avoiding the tilting phenomena inherent to the anatomical functioning of the joint, and reinforcing the primary stability of said prosthesis.

In addition, and according to another characteristic of the invention, the stem continuing the face of the seat intended to come into contact with the bone of the glenoid cavity is provided with holes in such a way as to promote secondary fixation of the prosthesis by bone growth or migration.

According to the invention, the means ensuring the cooperation and fixation of the female insert with the plate of the seat consist of blind holes able to cooperate with pins of suitable diameter issuing from the lower part of the female insert, or of recesses formed at the internal periphery of and inside the seat and able to cooperate with projections of corresponding dimensions issuing from the base of the female insert.

According to another characteristic of the invention, the through-holes in the plate are each provided with an expansion socket which is able to cooperate with the anchoring screws and which is intended to immobilize said screw in the orientation chosen by the practitioner. This socket is itself provided with peripheral studs projecting from its outer envelope, said studs being intended to be received in corresponding recesses formed in a ring welded inside said holes.

The manner in which the invention is realized and the advantages which derive from it will be better appreciated from the following illustrative embodiment which is given as a nonlimiting example and with reference to the attached figures.

FIG. 1 is a diagrammatic perspective view of the seat of the glenoid cavity prosthesis according to the invention, provided with its anchoring screws.

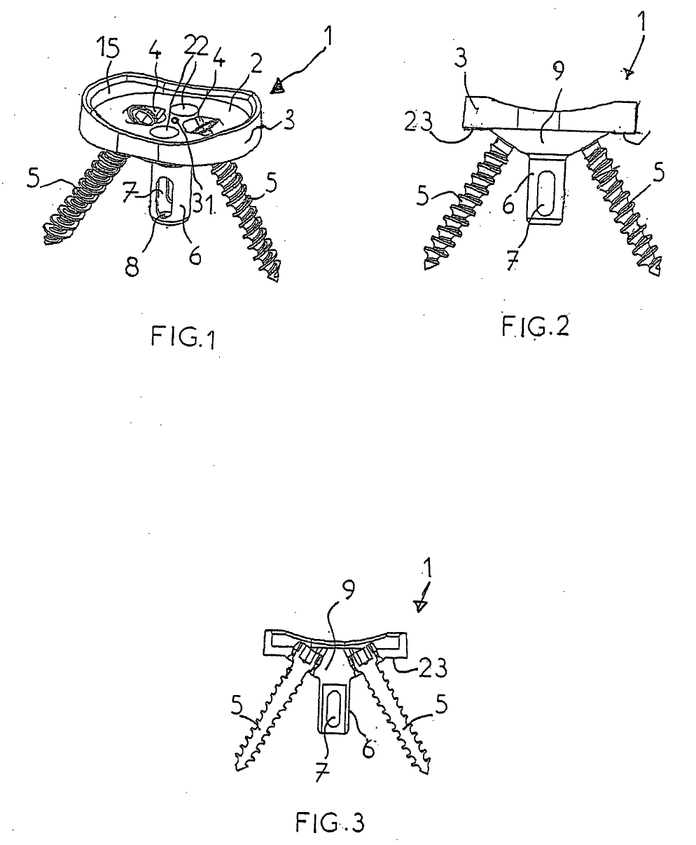

FIG. 2 is a side view of the seat from FIG. 1.

FIG. 3 is a diagrammatic cross-sectional view of the seat from FIG. 1.

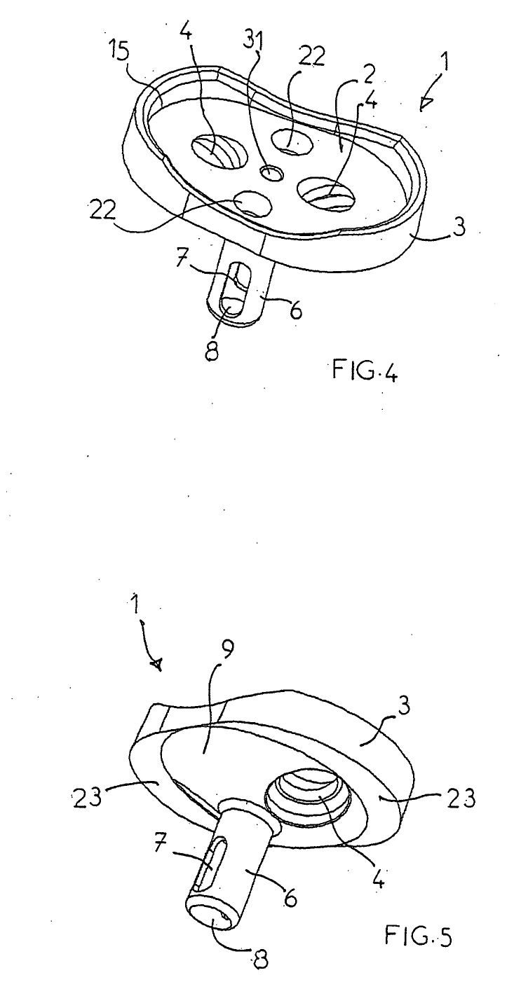

FIG. 4 is a diagrammatic perspective view of the seat according to the invention in a simplified version and seen from above.

FIG. 5 is a diagrammatic perspective view of the seat from FIG. 4 seen from below.

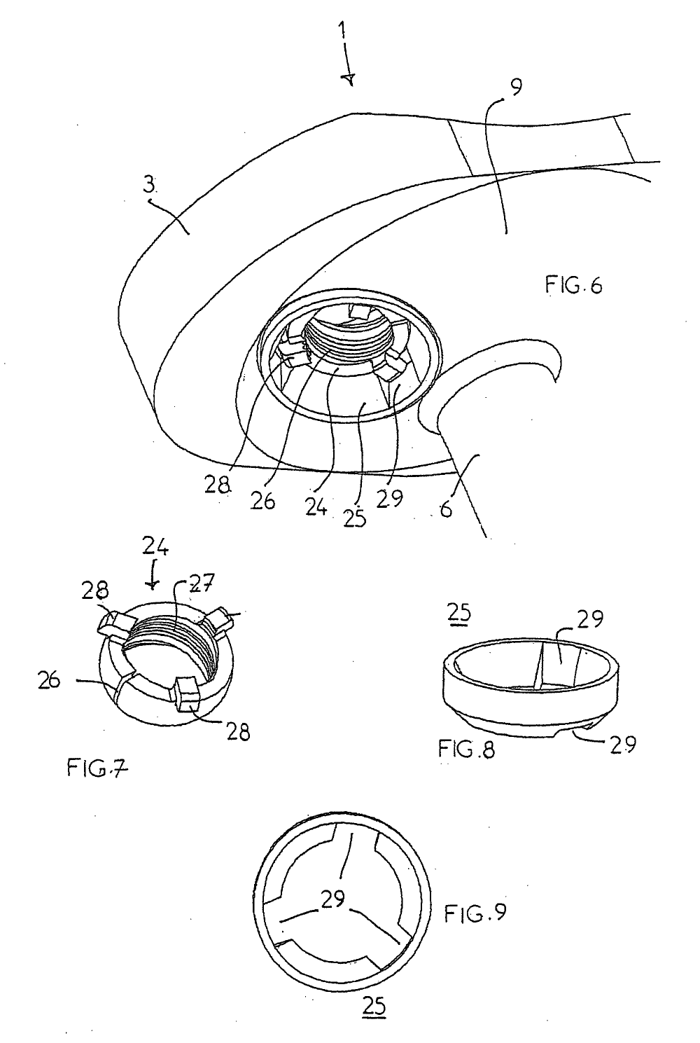

FIG. 6 is a detail from FIG. 5.

FIG. 7 is a diagrammatic perspective view of an expansion socket according to the invention.

FIG. 8 is a diagrammatic perspective view of a ring according to the invention, intended to cooperate with the socket from FIG. 7, and of which FIG. 9 is a plan view.

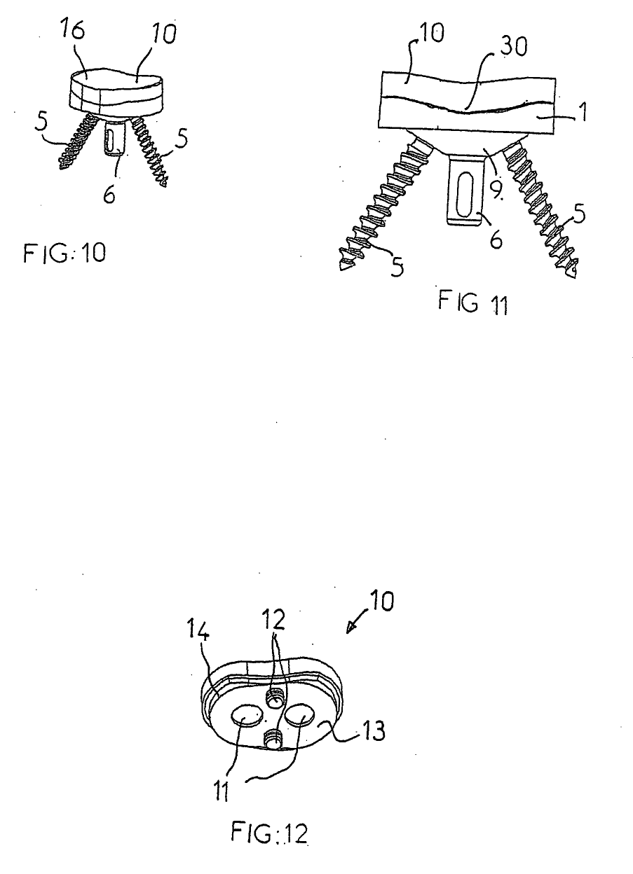

FIG. 10 is a diagrammatic perspective view of the seat of the glenoid cavity prosthesis according to the invention, provided with a female insert.

FIG. 11 is a side view of FIG. 10.

FIG. 12 is a diagrammatic perspective bottom view of the female insert according to the invention.

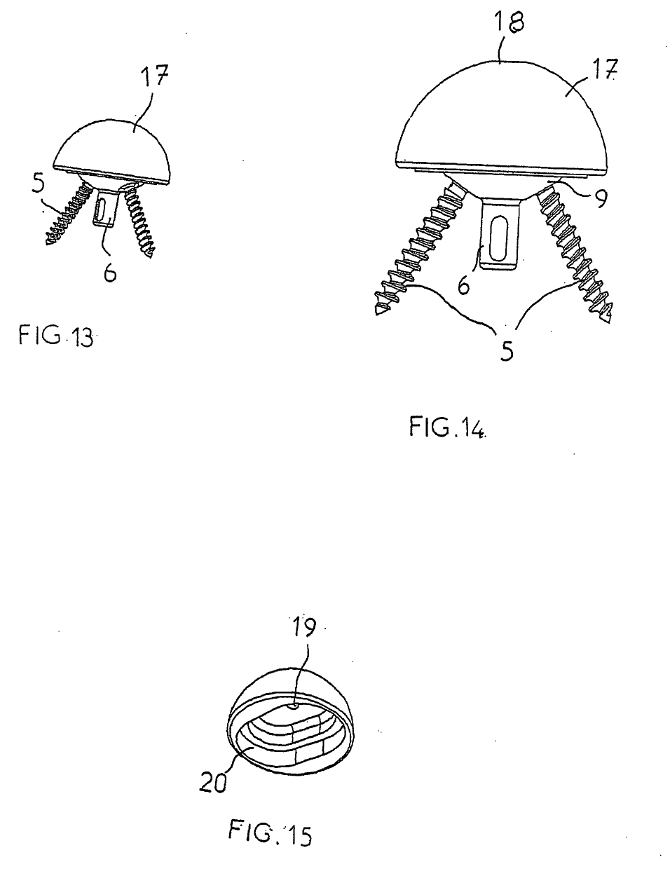

FIG. 13 is a diagrammatic perspective view of the seat of the glenoid cavity according to the invention, provided with a male insert according to the invention.

FIG. 14 is a side view of FIG. 13.

FIG. 15 is a diagrammatic perspective bottom view of the male insert according to the invention.

The prosthesis for the glenoid cavity of the scapula according to the present invention has thus been shown in FIGS. 1 to 6. Basically, this prosthesis consists of a seat (1) comprising a plate (2) which is intended to be fixed by anchoring in the osseous glenoid cavity of the patient to be treated, after preparation, and in particular reaming, by the practitioner.

This seat (1) thus has a substantially plane front or upper face, called the plate (2), and a lower face intended to come into contact with the bone. According to one characteristic of the invention, this lower face is bulged (9), as can be seen from FIGS. 2 and 5. In addition, the lower face has two flat areas (23) situated on either side of the bulged shape (9). This particular shape helps give a certain stability to the prosthesis when it is being fitted. Indeed, the combination of the bulged shape (9) and of the flat areas cooperating with reamed zones previously formed in the osseous glenoid cavity makes it possible, in the first instance, to optimize the contact between bone and implant and, in addition, to avoid the phenomena of tilting of the seat, and thus of the prosthesis, which phenomena are inherent to the actual functioning of the shoulder joint.

This stability is additionally optimized by the presence of a stem (6) oriented substantially perpendicularly with respect to the seat (1) and continuing said bulged part (9). This stem (6) is intended to be positioned in a bore which the practitioner has formed beforehand in the osseous glenoid cavity.

According to one characteristic of the invention, this stem is provided with a through-hole (7) along its generatrix and a through-hole (8) at its lower end. These through-holes are intended to promote the holding of the prosthesis by secondary fixation (bone migration and growth).

By virtue of its structure, this seat makes it possible to preserve as much as possible of the bone of the glenoid cavity.

Consequently the seat (1) is anchored in the glenoid cavity by means of two anchoring screws (5). For this purpose, the plate (2) of the seat is provided with two inclined through-holes (4) in order to promote the passage of the screws (5) around an immobilizing system described below in more detail.

These screws can be oriented by approximately 12° relative to the axis of the holes (4) in order to allow the practitioner to place them in the supraglenoid and infraglenoid tubercles.

The holes (4) are inclined by an angle value of about 30° relative to the plane containing the upper face of the plate (2) of the seat. The screws, advantageously made of titanium alloy or stainless steel, are self-drilling, self-tapping and retrocutting. The conical core of the rods and the cylindrical threading with which they are provided promote anchoring in the bone, and in particular in the glenoid cavity in question.

The screw head is conical and has a quadruple threading intended to cooperate with the immobilizing system. More precisely, the screw head is provided with four parallel threads opening out at the lower base of the head at an angle of 90° between two consecutive threads. These four threads are intended to cooperate with a corresponding quadruple threading formed in the inner wall of the immobilizing system, described below in more detail. By means of this quadruple threading, the practitioner is aided in his task of introducing the screws, insofar as the number of possibilities of locking the screw to the immobilizing system is multiplied by sixteen.

The immobilizing system, to which reference has been made above, is formed by an expansion socket (24) and a ring (25). This socket is slit (26) along one of its generatrices, so as to allow its expansion. It has a quadruple internal threading (27) intended to cooperate with the quadruple threading of the heads of the screws (5). This being the case, screwing of the screw into the socket (24) spreads the latter apart, and it is then immobilized, in the area of the ring (25) welded in each of the holes (4) situated inside the seat, at the orientation desired by the practitioner. In addition, the base of the socket (24) is provided with three studs (28) which project from the envelope of the ball, are periodically distributed and are intended to be received in corresponding recesses (29) of said ring (25). This being the case, by virtue of the cooperation between the studs (28) and the recesses (29), the socket (24) cannot turn during fitting of the screws (5) by the practitioner.

According to the invention, the plate (2) of the seat (1) is provided at its periphery with a rim (3) of particular shape, since it is in the form of a morse cone, or, more exactly, a truncated morse cone. It is intended to cooperate with the male insert described below in more detail.

The female insert (10) according to the invention has been shown in FIGS. 10 to 12. It is intended to cooperate with the seat described above. This insert is made in particular of polyethylene. It has a minimum thickness of 5 mm in the area of its thinnest zone. It is provided, on its lower face (13), with two positioning and fixing pins (12) which are intended to cooperate, by clipping action, with blind holes (22) formed in the plate (1) of the seat.

This female insert (10) thus clips onto said seat, on the one hand through the cooperation of the pins (12) with the holes (22), and, on the other hand, through the cooperation between the internal peripheral edge (14) of the insert on the inner wall (15) of the peripheral rim (3) of the seat. In addition, the antero-posterior zones (30) of the insert have increased material, making it possible to increase the life of the implant. This is because, given the stressing of the insert in this area, inherent to the mode of functioning of the joint, greater wear is generally observed at this level. Finally, the lower face (13) of the insert is provided with cutouts (11) guaranteeing the passage of the screw heads (5). The upper face (16) of the insert is of concave shape, in particular of anatomical shape, able to cooperate with the humeral head or with a humeral implant in the context of the use of a total shoulder prosthesis.

The use of a male insert in relation with the seat according to the invention has been shown in FIGS. 13 to 15. This male insert (17) is of hemispherical shape, its pole (18) being provided with a hole (19) intended to allow it to be fitted on the seat (1) by a suitable ancillary device, or to be removed therefrom.

This male insert (17) is made of stainless steel, for example. It is fixed on the seat by conical engagement on the morse cone formed at the periphery of the rim (3) of said seat. More precisely, it is engaged in the area of its lower internal wall (20), as will be seen in FIG. 15. This fixation is additionally secured by means of a screw passing through the hole (19) and being screwed into a threaded hole (31) formed on the plate (2) of the seat (1).

All the benefits of the prosthesis according to the invention will be appreciated, in particular its modular character which allows the practitioner, using a standard seat, to use an insert adapted to the trauma or to the pathology, said insert being able to be positioned easily and reliably, avoiding the disadvantages associated with the phenomena of migration and instability, both of the seat and of the inserts.

1. Prosthesis for the glenoid cavity of the scapula, comprising a seat (1), intended to be fixed to the glenoid cavity of the patient to be treated by means of anchoring screws (5), said seat (1) comprising a plate (2) able to receive a male insert (17), and said seat being able to receive a female insert (10), said inserts (10, 17) being intended to cooperate with the humeral head of the shoulder joint in question or a humeral implant fitted in the area of said joint, characterized:
in that the outer face of the peripheral rim (3), with which the seat (1) is provided, is configured as a truncated morse cone able to cooperate with a male insert (17) provided with a bearing surface of complementary shape,
and in that the plate (2) of the seat (1) is provided with means (22) intended to cooperate with a female insert (10) in order to ensure that the latter is fixed by clipping into it.

2. Prosthesis for the glenoid cavity of the scapula according to claim 1, characterized in that the plate (2) is provided with two through-holes (4), each of them inclined by an angle value of between 20° and 35° relative to the general plane of the plate, and being intended to permit the passage of anchoring screws (5) into the glenoid cavity.

3. Prosthesis for the glenoid cavity of the scapula according to claim 1, characterized in that the rear face of the seat, intended to cooperate with the glenoid cavity, is bulged (9) and is provided with a stem (6) intended to be implanted in said glenoid cavity.

4. Prosthesis for the glenoid cavity of the scapula according to claim 3, characterized in that the rear face of the seat (1), intended to cooperate with the glenoid cavity, has two flat areas (23) situated at end positions relative to the bulged part (9), and thus situated on either side of the bulged zone and intended to bear inside the glenoid cavity.

5. Prosthesis for the glenoid cavity of the scapula according to claim 3, characterized in that the stem (6) continuing the rear face of the seat is provided with holes (7, 8) in such a way as to promote secondary fixation of the prosthesis by bone growth or migration.

6. Prosthesis for the glenoid cavity of the scapula according to claim 1, characterized in that the means ensuring the cooperation and fixation of the female insert (10) with the plate (2) of the seat (1) consist of blind holes (22) able to cooperate with pins (12) of suitable diameter issuing from the lower part of the female insert (10), or of recesses formed at the internal periphery of and inside the seat and able to cooperate with projections of corresponding dimensions issuing from the base of the female insert.

7. Prosthesis for the glenoid cavity of the scapula according to claim 1, characterized in that the holes (4) in the plate (2) are each provided with an expansion socket (24) which is able to cooperate with the anchoring screws (5) and which is intended to immobilize said screw in the orientation chosen by the practitioner.

8. Prosthesis for the glenoid cavity of the scapula according to claim 1, characterized in that the anchoring screws (5) are self-drilling, self-tapping and retrocutting.

9. Prosthesis for the glenoid cavity of the scapula according to claim 7, characterized in that the ball (24) is provided with peripheral studs (28) projecting from its outer envelope, said studs (28) being intended to be received in corresponding recesses or indentations (29) formed in a ring (25) welded in said holes (4).

10. Prosthesis for the glenoid cavity of the scapula according to claim 6, characterized in that the female insert (10) is provided with two fixing pins (12) intended to clip into the holes (22) formed in the plate of the seat (1), and in that its lower face is provided with cutouts (11) intended to permit the passage of the heads of the anchoring screws (5).

11. Prosthesis for the glenoid cavity of the scapula according to claim 6, characterized in that the female insert (10) has an increased thickness in the area of its antero-posterior zones (30).

12. Prosthesis for the glenoid cavity of the scapula according to claim 1, characterized in that the anchoring screws (5) can be oriented by an angle value of approximately 12° inside the through-holes (4) of the plate (2).
